(19) United States
(12) Patent Application Publication
(10) Pub. No.: US 2024/0050497 A1
(43) Pub. Date: Feb. 15, 2024

(54) USE OF A COMPOSITION COMPRISING MICROORGANISMS TO INCREASE THE INTESTINAL PRODUCTION OF BUTYRIC ACID, FOLIC ACID OR NIACIN AND/OR DECREASE THE INTESTINAL PRODUCTION OF SUCCINIC ACID

(71) Applicant: ALFASIGMA S.P.A., BOLOGNA (IT)

(72) Inventors: Simone Domenico GUGLIELMETTI, MILANO (IT); Ruggero ROSSI, MILANO (IT); Walter FIORE, TREZZANO ROSA (IT); Andrea BIFFI, URGNANO (IT)

(21) Appl. No.: 18/493,221

(22) Filed: Oct. 24, 2023

Related U.S. Application Data
(63) Continuation of application No. 14/916,961, filed on Mar. 4, 2016, now Pat. No. 11,839,634.

(30) Foreign Application Priority Data
Sep. 6, 2013 (IT) MI2013A001467

(51) Int. Cl. A61K 35/747; A23L 33/135; A23L 33/21; A61P 1/00; A61K 9/00; A61K 9/48

(52) U.S. Cl. CPC A61K 35/747; A23L 33/135; A23L 33/21; A61P 1/00; A61K 9/0053; A61K 9/48; A23V 2002/00; A23V 2400/165; A61K 2035/11

(57) ABSTRACT

Use of a composition comprising bacteria to increase the intestinal production of butyric acid, folic acid or niacin and/or to decrease the intestinal production of succinic acid.

Moreover, the present invention relates to the use of said composition for the treatment and/or prevention of an intestinal butyrate- and/or succinate-dependent pathological condition. In particular, for the treatment and/or the prevention of intestinal inflammation, diarrhoea, ulcerative colitis or intestinal colopathies.

### CROSS REFERENCE TO RELATED APPLICATIONS

The present application is a divisional of U.S. patent application Ser. No. 14/916,961 filed on Mar. 4, 2016, incorporated herein by reference, which, in turn, is a 371 National Stage application of International PCT Application No. PCT/IB2014/064285, filed on Sep. 5, 2014, which, in turn, claims benefit of priority to Italian Patent Application No. M12013A0014657 filed on Sep. 6, 2013, the disclosure of each of which is incorporated by reference in their entirety.

### FIELD

The present invention relates to the use of a composition comprising bacteria in order to increase the intestinal production of butyric acid, folic acid or niacin and/or to decrease the intestinal production of succinic acid. Moreover, the present invention relates to the use of said composition for the treatment and/or prevention of an intestinal butyrate- and/or succinate-dependent pathological condition, in particular, for the treatment and/or prevention of intestinal inflammation, diarrhoea, ulcerative colitis or intestinal colopathies.

### REFERENCE TO SEQUENCE LISTING

Further, the computer readable form of the sequence listing of the XML text file P2413-USD-2023-10-15.xml, created on Oct. 15, 2023, with a size of 2,803 bytes measured on Windows Server 2019 Datacenter, is incorporated herein by reference in its entirety.

### BACKGROUND

Intestinal microbiota, also known by the by now obsolete term of intestinal flora, is the whole of the microorganisms, prevalently consisting of bacteria, residing in the intestine and in symbiosis with the body of the host. The intestinal microbiota is a highly complex ecosystem and the condition of equilibrium among the different microorganisms making up the intestinal is fundamental in order to ensure the body's well-being and health, since the microbiota significantly conditions the development and the homeostasis of the intestinal mucosa of the host individual.

In other words, the intestinal microbiota represents a veritable organ. In fact, qualitative and/or quantitative modifications in the intestinal microbiota of an individual, or so-called disbiosis or dismicrobism, can result in the loss of the intestinal homeostasis, which in turn can condition the etiopathogenesis of a large number of pathologies.

For the purpose of treating a condition of intestinal disbiosis, or, in any case, for the purpose of maintaining the homeostasis of the intestinal microbiota, people often take substances that are defined as probiotics, or, according to the definition of the FAO/WHO, “live microorganisms which, when administered in adequate amounts, confer a health benefit on the host”.

Similarly, the effectiveness of paraprobiotics for health has also been demonstrated; these are defined as “non-viable microbial cells (intact or broken) or raw cellular extracts which, when administered in adequate amounts (orally or topically), confer a health benefit on the host” (Taverniti and Guglielmetti, 2011).

It is clear that the beneficial activities of a microorganism will vary depending on the composition thereof and, in fact, these are often strain-specific activities.

On the basis of the above considerations, there continues to be a felt need to determine potential new health-promoting and/or therapeutic effects of microorganisms, in particular those included in a probiotic or in a paraprobiotic, in order to broaden the applications of use.

For example, there continues to be a greatly felt need in the art to identify microorganisms capable of modulating the intestinal amount of substances that are particularly beneficial and therapeutic for the body, such as butyric acid, folic acid and nicotinic acid.

Butyric acid is a short-chain fatty acid which is physiologically formed in the colon of humans as a result of the fermentation of dietary fibre by the microbiota.

Butyric acid is the principal source of energy for colon cells (colonocytes) and is therefore a nutrient that is essential for the human body.

At the intestinal level, butyric acid performs various important functions, e.g.: it stimulates the turnover and physiological maturation of colonocytes; it plays a key role in maintaining the integrity of the mucosa and in processes of repairing intestinal lesions; it stimulates the reabsorption of water and sodium in the colon; and it contributes to lowering the intestinal pH, creating an environment that is unfavourable to the development of pathogenic bacteria.

A deficiency of butyric acid can cause inflammatory colitis in humans.

Succinic acid is likewise a short-chain organic acid, of the bicarboxylic type.

It is considered ulcerogenic and can cause serious damage to the mucosa.

Therefore, an increase in the amount of succinic acid (succinate) is harmful to human health.

Folic acid (vitamin B9, or M or folacin) is a very important vitamin for the whole population, in particular in adults over 50 years of age and in women of a fertile age, because it intervenes (directly or, most of the time, by decreasing the plasma levels of homocysteine) in many vital processes such as DNA synthesis, repair and methylation.

A deficiency of folic acid can lead to macrocytic anaemia, which may be accompanied by leukopaenia and thrombocytopaenia, skin and mucosa alterations and gastrointestinal disorders (malabsorption and diarrhoea).

Niacin (or vitamin PP or vitamin B3), i.e. nicotinic acid and nicotinamide, is important because, among other things, it is the essential component of the coenzymes NAD and NADH and a deficiency thereof causes a pathology known as pellagra. Generally, this pathology begins with problems in the gastrointestinal system, which are then compounded by a photosensitizing dermatitis, mental disorders with fatigue, depression and memory disorders.

### SUMMARY

The present invention responds to the needs of the prior art described above with a composition comprising microorganisms, preferably bacteria of the genus Lactobacillus species paracasei, capable of (directly and/or indirectly) increasing, in an individual that takes it, the intestinal production of butyric acid, folic acid, niacin and/or salts thereof.

Furthermore, the Applicant has found, wholly unexpectedly, that a composition comprising microorganisms, preferably of the genus Lactobacillus species paracasei, is capable of (directly and/or indirectly) decreasing the intestinal production of succinic acid and/or salts thereof.

Therefore, the composition of the present invention is particularly advantageous for the treatment and/or prevention of intestinal butyrate- and/or succinate-dependent pathological conditions.

Further advantages of the present invention will be more apparent from the detailed description that follows and from the examples which, however, have only a demonstrative, non-limiting purpose.

### BRIEF DESCRIPTION OF THE DRAWINGS

To enable a better understanding of the detailed description, FIGS. 1-5 have been appended hereto:

FIG. 1.1 shows the result of the statistical analysis demonstrating the increase in the population of bacteria of the genus Coprococcus before and after treatment with the composition of the present invention (A) and the decrease thereof, in contrast, before and after treatment with the placebo (B);

FIG. 1.2 shows the result of the statistical analysis demonstrating the decrease in the population of bacteria of the genus Blautia before and after treatment with the composition of the present invention (A) and the increase thereof, in contrast, before and after treatment with the placebo (B);

FIG. 2.1 shows the increase in the population of bacteria of the genus Coprococcus (dark grey) and the decrease in the population of bacteria of the genus Blautia (light grey) before and after treatment with the composition of the present invention;

FIG. 2.2 shows the percentage increase in the population of bacteria of the genus Coprococcus (dark grey) and the percentage decrease in the population of bacteria of the genus Blautia (light grey) before and after treatment with the composition of the present invention (A) and the percentage decrease in the population of bacteria of the genus Coprococcus (dark grey) and the percentage increase in the population of bacteria of the genus Blautia (light grey) before and after treatment with the placebo (B);

FIG. 3 shows the result of the statistical analysis which demonstrates the increase in the metabolism of nicotinic acid before and after treatment with the composition of the present invention and the decrease thereof before and after treatment with the placebo;

FIG. 4 shows the result of the statistical analysis which demonstrates the increase in the biosynthesis of folic acid before and after treatment with the composition of the present invention and an absence of any modifications, in contrast, before and after treatment with the placebo; and

FIG. 5 shows a schematic of the probiotic dietary intervention carried out in accordance with a design crossover.

### DETAILED DESCRIPTION

The present invention relates to the use of a composition comprising microorganisms, preferably at least one bacterium of the genus Lactobacillus species paracasei, to increase the direct and/or indirect intestinal production of butyric acid and/or salts thereof, and/or folic acid and/or salts thereof, and/or niacin and/or salts thereof and/or to decrease the direct and/or indirect intestinal production of succinic acid and/or salts thereof.

In the context of the present invention, intestinal production means the release, into the environment, of any molecule produced by primary or secondary metabolism by any intestinal microorganism in any region of the intestine.

Moreover, the composition of the present invention can also be used to reduce the intestinal proliferation of pathogenic microorganisms, and/or to promote the integrity of the intestinal mucosa, and/or to promote the processes of repair of intestinal lesions, preferably by increasing the direct and/or indirect intestinal production of butyric acid and/or salts thereof and/or by decreasing the direct and/or indirect intestinal production of succinic acid and/or salts thereof.

Some pathogenic microorganisms particularly sensitive to the composition of the present invention are, for example, enterohaemorrhagic Escherichia coli, Listeria monocytogenes, Clostridium difficile, Pseudomonas aeruginosa and Salmonella spp.

The above-described uses of the composition of the present invention are intended both for a healthy individual and an individual with a pathological intestinal condition. In particular, in the case of a healthy individual, the composition of the invention performs in that individual, following intake, an action of maintaining the homeostasis of the microbiota and/or of preventing an alteration thereof, and is thus also definable as a probiotic composition (or probiotic).

A further aspect of the present invention relates to the medical use of the composition comprising microorganisms, preferably at least one bacterium of the genus Lactobacillus species paracasei, for the treatment and/or prevention of an intestinal butyrate- and/or succinate-dependent pathological condition.

In the context of the present invention, intestinal butyrate- and/or succinate-dependent pathological condition means a pathological condition that is sensitive to treatment with butyric acid and/or salts thereof and/or treatment with succinic acid and/or salts thereof. Examples of said pathologies are: diarrhoea, intestinal inflammation, ulcerative colitis, gastric atrophy, intestinal diverticula, stenosis, obstructions and diabetic neuropathy.

In a particularly preferred embodiment of the present invention, the composition comprises the bacterial strain Lactobacillus paracasei DG.

The bacterial strain Lactobacillus paracasei DG was deposited by SOFAR S.p.A. with the National Collection of Microorganism Cultures of the Pasteur Institute (CNCM) in Paris on May 5, 1995, with the deposit number CNCM I-1572. CNCM has an address of 25, rue du Docteur Roux 75724 Paris Cedex 15. Initially, the name of the deposited strain was Lactobacillus casei DG sub.casei.

In a further embodiment of the invention, the direct and/or indirect increase in the intestinal production of butyric acid and/or salts thereof, and/or of folic acid and/or salts thereof, and/or of niacin and/or salts thereof and/or the direct and/or indirect decrease in the intestinal production of succinic acid is ascribable to the intestinal microbiota, preferably bacteria of the genus Coprococcus and/or Blautia.

In the particularly preferred embodiment of the invention, the direct and/or indirect increase in the intestinal production of butyric acid and/or salts thereof is ascribable to bacteria of the genus Coprococcus, and/or the direct and/or indirect decrease in the intestinal production of succinic acid is ascribable to bacteria of the genus Blautia.

Therefore, the composition comprising microorganisms, preferably at least one bacterium of the genus Lactobacillus species paracasei, more preferably the bacterial strain Lactobacillus paracasei DG, can also be used to modify the density of the bacterial population of the genus Coprococcus and/or Blautia in the intestinal microbiota, preferably so as to induce an increase in the bacterial population of the genus Coprococcus and/or a decrease in the bacterial population of the genus Blautia. In other words, intake of the composition of the present invention modifies the amount of bacteria of the genus Coprococcus and/or Blautia within the intestinal microbiota. In particular, the bacteria of the genus Coprococcus increase and/or the bacteria of the genus Blautia decrease following intake of said composition.

The composition used in the present invention comprises said microorganism, preferably said at least one bacterium of the genus Lactobacillus species paracasei, in live or dead form, as a lysate or extract. In one embodiment of the invention, the composition comprises about 15-billion colony forming units (CFU) of bacteria, preferably 20-25 billion CFU of bacteria.

Preferably, the composition is formulated for oral administration. In particular, the composition is formulated in solid form, preferably in the form of pills, capsules, tablets, granular powder, hard capsules, water-soluble granules, sachets or pellets.

Alternatively, the composition of the invention is formulated in liquid form, for example as a syrup or beverage, or is added to a food, for example to a yogurt, cheese or fruit juice.

Alternatively, the composition of the invention is formulated in a form capable of exerting an action topically, for example as an enema.

In one embodiment of the invention, the composition further comprises excipients generally accepted for the production of probiotic and/or pharmaceutical products.

In a further embodiment of the invention, the composition of the invention can be enriched with vitamins, trace elements such as zinc and selenium, enzymes, prebiotic substances such as fructo-oligosaccharides (FOS), galacto-oligosaccharides (GOS), inulin, guar gum or combinations thereof. Preferably, for the purposes of the uses of the present invention, the composition is taken once a day, more preferably upon awakening. Alternatively, it can also be taken in the evening, preferably after meals.

### Example

Treatment.

A randomized, double-blind, placebo-controlled crossover dietary intervention study was conducted on healthy individuals.

Volunteers were recruited in accordance with the following criteria: inclusion criteria: healthy men and women, ranging in age between 18 and 55 years who gave their informed consent;

exclusion criteria: antibiotic treatment in the month preceding the first examination; episodes of viral or bacterial enteritis in the 2 months preceding the first examination; gastric or duodenal ulcers in the 5 years preceding the first examination; pregnancy or breastfeeding; recent or presumed cases of alcoholism and drug intake; other conditions of non-compliance with the study protocol.

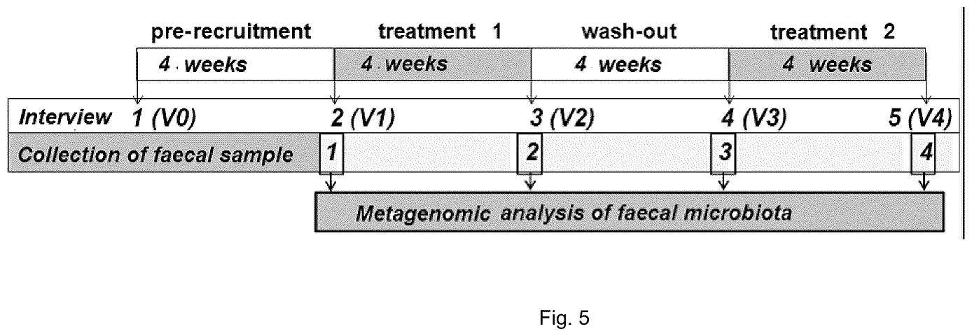

The probiotic dietary intervention was carried out in accordance with a design crossover, as schematized in FIG. 5.

In the pre-enrolment phase (4 weeks) the volunteers followed their usual diet, without consuming probiotic fermented milk products (traditional yogurt was thus permitted), probiotic dietary supplements, or prebiotic dietary supplements.

At the end of the pre-enrolment period, the volunteers were randomized to receive one capsule per day of a probiotic or placebo for 4 weeks.

By way of example, Enterolactis Plus was used as the probiotic to be administered; it consists in 420 mg capsules containing 24 billion CFU (colony forming units) of Lactobacillus paracasei strain DG.

The placebo consisted in capsules identical in appearance to the probiotic ones, obviously devoid of the probiotic agent.

The flavour and colour of the active substance (i.e. the probiotic) and the placebo were identical.

The product was taken in the morning on an empty stomach, at least ten minutes before breakfast or, if forgotten, in the evening before going to bed and in any case at least two hours after the last meal.

After the first four weeks of treatment, the volunteers went through a four-week wash-out period identical to the pre-enrolment period.

At the end of the wash-out period, the volunteers took one capsule per day of Enterolactis Plus or placebo for four weeks in accordance with the crossover design described above.

In summary, the study involved 4 phases, each of which lasting 4 weeks: Pre-recruitment phase: the individuals underwent neither treatment A nor treatment B.

Treatment 1: the individuals underwent treatment A or treatment B.

Wash-out: the individuals underwent neither treatment A nor treatment B

Treatment 2: the individuals underwent treatment B or treatment A.

Treatments A and B can be the composition of the present invention, in the specific example Enterolactis plus, or else the placebo. At the start of the treatment, it was not known what the individual was taking; only at the end of the treatment, when the blind was broken, was the intake sequence known.

Examinations and Sample Collection.

Each volunteer was initially instructed as to the entire procedure to be followed, which involved a total of 5 meetings per volunteer.

During the first meeting, informed consent was obtained along with the volunteer's personal data. The volunteer also received general information about how the study was to be carried out and was instructed about the changes in the diet to be applied in the subsequent 4 weeks of pre-enrolment (prohibition from consuming the previously specified products). After 4 weeks, the volunteer went to the second meeting with a faecal sample (sample TO), collected during the previous 24 hours in a special container handed over during the first meeting.

To ensure optimal preservation, the faecal samples were stored at room temperature and delivered to the laboratory within 24 hours.

During the second meeting, moreover, the volunteer was given the probiotic product (or placebo) to be taken during the next 4 weeks. Moreover, the volunteer was instructed as to how to take the product.

At the end of the 4 weeks of taking the product (or placebo), the volunteer went to the third meeting with another faecal sample (sample T1) collected during the previous 24 hours.

During the third meeting, the volunteer completed a questionnaire on the possible effects, both positive and undesirable ones, deriving from consumption of the product.

The volunteer was then instructed about the next 4 weeks, during which he or she again did not take the previously mentioned products.

At the end of these 4 weeks, the volunteer went to the fourth meeting with a faecal sample (sample T2) and received the probiotic product (or placebo) to be taken during the next 4 weeks.

Finally, after 4 weeks of taking the product (or placebo), the volunteer went to the fifth meeting to deliver the last faecal sample (sample T3).

During this last meeting, the volunteer has completed a questionnaire analogous to the one received during the third meeting.

All the faecal samples collected were stored at −20° C. for no more than 7 days before being subjected to analysis of the microbiota.

Analysis of Faecal Microbiota

The faecal microbiota was evaluated by analyzing the nucleotide sequence of portions of the gene encoding the 16S rRNA bacterial ribosomal subunit.

More specifically, a metagenomic strategy was adopted; it consists in short in the following steps:
extracting, quantifying and normalizing the metagenomic DNA from the faecal samples;
amplifying the V3 hypervariable region of the bacterial gene encoding the 16S rRNA by PCR;
quantifying the PCR products;
sequencing the amplification products;
bioinformatically analyzing the sequences.

The procedures according to steps 1 and 3 are techniques that are well known in the art and they are thus performed with the protocols commonly used in this field. For example, the methods described in laboratory manuals such as those by Sambrook et al. 2001, or Ausubel et al. 1994.

Step 2 of amplifying the V3 region of the 16S ribosomal RNA genes was performed by means of the DNA amplification technique known as PCR, using Probio_Uni 5′-CCTACGGGRSGCAGCAG-3′ (SEQ ID NO: 1) and Probio_Rev 5′-ATTACCGCGGCTGCT-3′ (SEQ ID NO: 2) as oligonucleotides (primers).

In particular, the pair of primers SEQ ID NO: 1 and 2 2 amplifies the V3 region of the 16S rRNA gene.

Step 4 can be performed with the techniques known in the art for this purpose, for example techniques based on the Sanger method, pyrosequencing or the Ion Torrent Fusion Primers sequencing method used in the specific example of the present invention according to the protocol described in the materials and methods section of the scientific article by Milani et al. (2013).

In the case of the Ion Torrent technique, the primers are designed and synthesized in such a way as to include, at the 5′ end, one of the two adaptor sequences used in this specific DNA sequencing technique. In this case, the adaptor sequences were SEQ ID NO: 1 and 2.

The conditions under which the PCR was performed are the following:
5 minutes at 95° C.;
30 seconds at 94° C., 30 seconds at 55° C., and 90 seconds at 72° C. for 35 cycles;
10 minutes at 72° C.

At the end of the PCR, the integrity of the amplificate was verified by electrophoresis.

Step 5 of the method, necessary for characterizing the microbial communities, can be carried out with numerous techniques presently known for this purpose. More specifically, use was made of: hierarchical clustering, taxonomic analysis and construction of phylogenetic dendrograms with heat maps according to the protocol described in the materials and methods section of the scientific article by Milani et al. (2013); more specifically, the analysis of sequence data was conducted using QIIME software.

Statistical Analysis of the Data

The statistical analysis was conducted using STATISTICA software (Statsoft Inc., Tulsa, OK, USA).

In order to reveal significant differences, the data were analyzed using both parametric (multivariate and univariate repeated-measures ANOVA) and non-parametric (Wald-Wolfowitz and Mann-Whitney) statistical methods. The normality of the data series (important assumption for ANOVA) was evaluated by means of the Shapiro—Wilk and Kolmogorov-Smirnov tests.

Results of the Treatment

The study was completed by a total of 22 individuals (11 females and 11 males).

Thirty-three individuals were initially enrolled, but 11 of them withdrew early for various reasons: intake of antibiotics (4), refusal to continue the study (1), frequent episodes of diarrhoea (1), intake of other probiotics during the study period (3), drastic change in eating habits (1), and seasonal influenza with episodes of diarrhoea (1).

Upon the conclusion of the study and completion of the analysis of the results of the two treatments, the blind was broken and it was seen that: treatment A is the active treatment, containing Lactobacillus paracasei DG; treatment B is the placebo, identical on the exterior to the active treatment, but devoid of lactobacilli.

When the data obtained from the study were analyzed, a high stability, from a taxonomic viewpoint, of the intestinal microbiota of the study participants was observed.

In fact, it was found that:
Two bacterial divisions of the 15 identified, namely, Bacteroidetes and Firmicutes, constitute over 90% of the sequences;
11 families of the 131 identified constitute over 90% of the sequences; and
20 genera of the 262 identified constitute over 90% of the sequences.

Moreover, this study confirmed that human intestinal microbiota at lower taxonomic levels (i.e. at the family and genus levels) is highly variable from one individual to another.

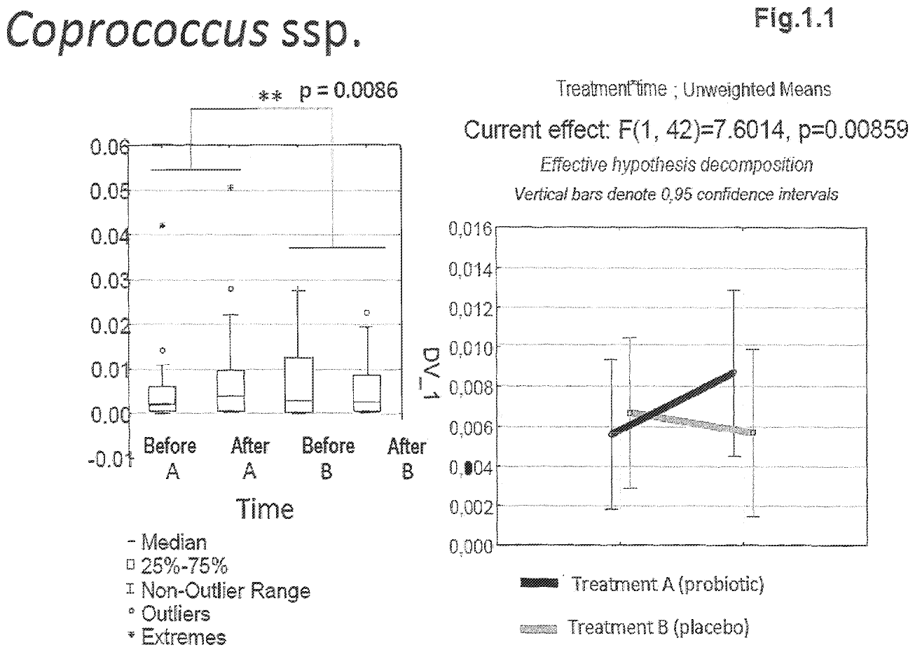

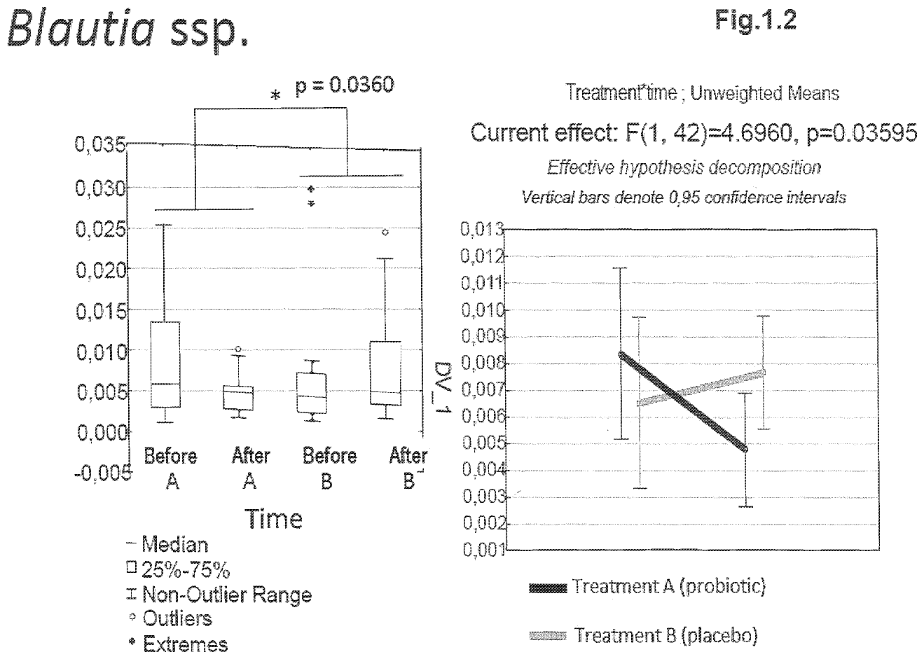

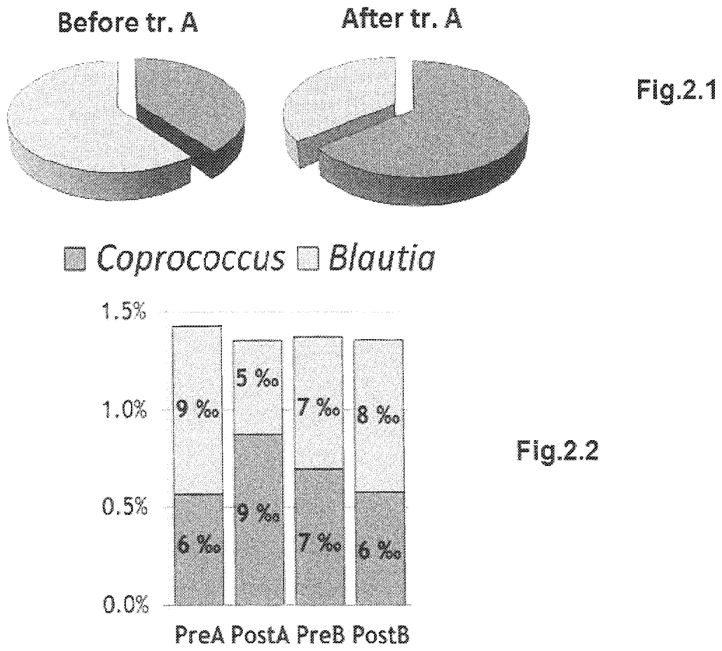

Therefore, the experimental evidence demonstrated the necessity of conducting, on a healthy population, crossover intervention trials in order to prevent the marked inter-individual variability from hiding the possible effects of the probiotic treatment or leading to false statistical positives. When the modifications induced in the intestinal microbiota by the two treatments were evaluated, a statistically significant difference emerged in terms of genera only in the group receiving the treatment with Lactobacillus paracasei DG (active treatment). More specifically, an increase in the genus Coprococcus was observed. In fact, as can be noted in FIGS. 1.1, 2.1 and 2.2, before and after treatment with Lactobacillus paracasei DG a statistically significant increase in coprococci was observed. In contrast, a moderate reduction thereof was seen in the group receiving the placebo treatment.

Moreover, after treatment with Lactobacillus paracasei DG, a statistically significant reduction in bacteria of the genus Blautia was observed. In contrast, a slight increase thereof was observed in the group receiving the placebo treatment (FIGS. 1.2, 2.1 and 2.2) Coprococci are among the main producers of butyrate at the intestinal level.

Butyrate is a fundamental compound at the intestinal level, since on the one hand it contributes to restoring the functional integrity of the intestinal mucosa and maintaining it over time, and on the other hand it has important anti-inflammatory effects, so much so that it is used as an adjuvant to dietary treatments for intestinal colopathies (e.g. chronic inflammatory intestinal diseases).

Moreover, an analysis of their genome reveals that these bacteria can use succinate as a fermentation substrate.

This information is fundamental, in consideration of the fact that members of the genus Blautia generate acetate and succinate as main end products of the fermentation of glucose.

Succinate is considered an ulcerogenic factor, capable, therefore, of exacerbating the condition of individuals with ulcerative colitis, since it is probably to blame for the mucosal damage present above all in the active phases of the disease.

In conclusion, following treatment with a probiotic, in this case following the administration of Lactobacillus paracasei DG, one observes an increase in the bacteria belonging to the genus Coprococcus and hence an increase in the intestinal concentration of butyrate.

At the same time, one observes a reduction in the concentration of succinate, which may be to blame for mucosal damage in individuals with ulcerative colitis, in a direct manner, because following treatment with the probiotic, in this case following the administration of Lactobacillus paracasei DG, there is a reduction in the bacteria belonging to the genus Blautia, and, in an indirect manner, because the increased population of coprococci is further able to decrease the concentration of succinate by using it as a substrate in their fermentation process.

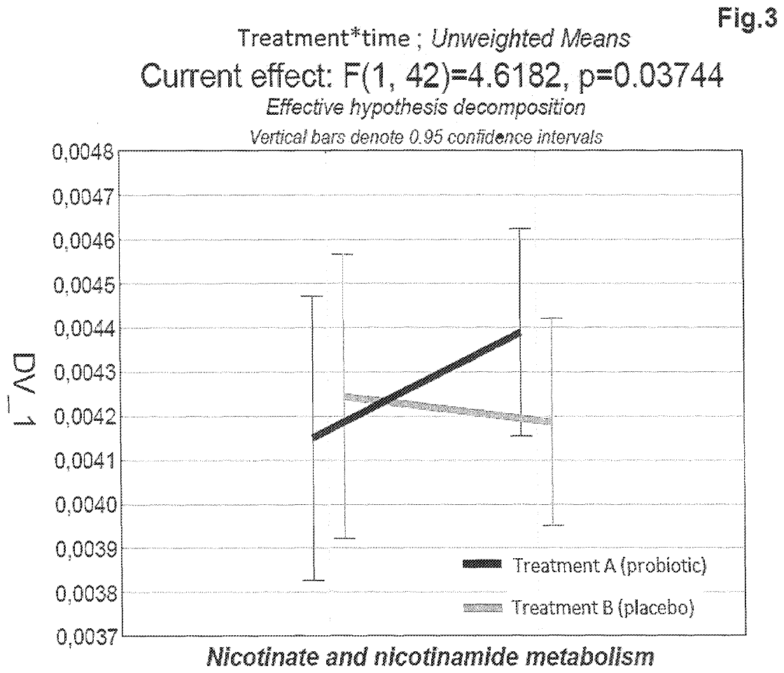

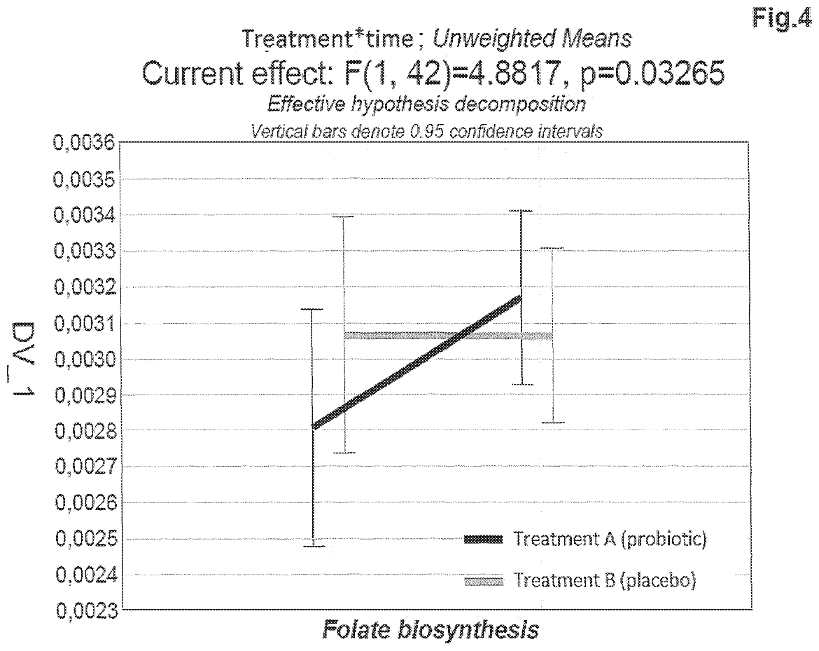

In conclusion, following treatment with the probiotic, in the specific example following the administration of Lactobacillus paracasei DG, there is an increase in the concentration of butyric acid in the faeces of individuals, with a simultaneous reduction in other organic acids, such as succinic acid. The data relating to the composition of faecal microbiota were used, finally, in a bioinformatic analysis aimed at a virtual reconstruction of the metagenome based on knowledge of the bacterial genomes (Okuda S, Tsuchiya Y, Kiriyama C, Itoh M, Morisaki H. Virtual metagenome reconstruction from 16S rRNA gene sequences. Nat Commun. 2012; 3:1203); in other words it was established in silico which potential genes are present and how abundantly in a given microbiota. This analysis made it possible to verify a putative increase in the encoding genes for the synthesis of folic acid and metabolism of nicotinic acid (FIGS. 3 and 4). These two molecules represent important vitamins for the human host (respectively named vitamin B9 and B3). Vitamin B9, in particular, represents a nutritional factor of primary importance, a deficiency of which, especially in specific physiological conditions such as pregnancy, can lead to serious health consequences. Treatment with the probiotic used in this study could therefore favor the ability of intestinal microbiota to produce folic acid (vitamin B9), with a consequent nutritional benefit for the human host.

1.-2. (canceled)

3. A method for treatment and/or prevention of an intestinal butyrate- and/or succinate-dependent pathological condition in a healthy subject, the method comprising
orally administering to the healthy subject, a composition comprising a bacterium Lactobacillus paracasei DG, Accession Number CNCM 1-1572, in 15-30 billion colony forming units (CFU), to prevent in the healthy subject the intestinal butyrate and succinate-dependent pathological condition,
wherein said intestinal pathological condition is intestinal diverticula.

4.-7. (canceled)

8. The method of claim 3, wherein said Lactobacillus paracasei DG, Accession Number CNCM I-1572 is a live or a dead bacterium, or a bacterial lysate or extract.

9. The method of claim 3, wherein said composition is in the form of pills, capsules, tablets, granular powder, hard capsules, water-soluble granules, sachets or pellets.

10. The method of claim 3, wherein said composition further comprises dietary fibres having prebiotic activity.

11. The method according to claim 10, wherein the dietary fibres having prebiotic activity comprise inulin and/or guar gum.

12. The method according to claim 10, wherein said composition further comprises vitamins, trace elements and/or enzymes.


SEQUENCE LISTING (2 sequences)

SEQ ID NO: 1 - DNA, 17 bp, synthetic construct
  source 1..17: mol_type = other DNA
cctacgggrsgcagcag

SEQ ID NO: 2 - DNA, 15 bp, synthetic construct
  source 1..15: mol_type = other DNA
attaccgcggctgct